(19) United States
(12) Patent Application Publication
(10) Pub. No.: US 2002/0123471 A1
(43) Pub. Date: Sep. 5, 2002

(54) LENTIVIRUS BASED VECTOR AND VECTOR SYSTEM

(76) Inventor: KLAUS UBERLA, MOEHRENDORF (DE)

Correspondence Address: CAROL M GRUPPI, MCCUTCHEN DOYLE BROWN & ENERSEN, THREE EMBARCADERO CENTER, SAN FRANCISCO, CA, 94111

(21) Appl. No.: 09/380,323

(22) PCT Filed: Mar. 3, 1998
(86) PCT No.: PCT/EP98/01191

(30) Foreign Application Priority Data
Jun. 3, 1997 (DK) 0238/97

(51) Int. Cl. A61K 48/00; A01N 63/00; C12N 15/86; C12N 7/01; C07H 21/04

(52) U.S. Cl. 514/44; 424/93.1; 435/456; 435/320.1; 435/235.1; 536/23.1

(57) ABSTRACT

The present invention relates to retroviral vectors which will infect and confer efficient gene transfer to non-dividing cells including the cells of the central nervous system. The vector system of the present invention is useful as a gene transfer vehicle for gene therapy, i.e. of the central nervous system.

The present invention relates to retroviral vectors which will infect and confer efficient gene transfer to non-dividing cells including the cells of the central nervous system. The vector system of the present invention is useful as a gene transfer vehicle for gene therapy, for example of the central nervous system.

### BACKGROUND OF THE INVENTION

Retroviral vectors allow efficient and stable transduction of a wide variety of cells. In contrast to most other viral vectors, genes transferred by retroviral vectors can persist in the absence of any viral protein due to integration into the host genome. Therefore, transduced cells are not rejected by antiviral immune responses. Retroviral vectors based on oncoviruses require cell division for efficient transduction (26,32). This severely limits the range of target cells and might prevent efficient in vivo gene therapy by retroviral vectors. To overcome this limitation, target cell division is induced in most gene transfer protocols. Peripheral blood lymphocytes, for example, are stimulated polyclonally and expanded with interleukin-2 in cell culture. Whether cells treated in such a way are still fully functional in vivo is largely unknown. The low transplantation efficiencies of transduced stem cell cultures could also be due to differentation of hematopoietic stem cells by culture conditions (2). In contrast to oncoviruses, lentiviruses can infect terminally differentiated cells (15,20) even if cell division is blocked by irradiation (35). As shown recently, retroviral vectors based on HIV-1 can transduce non-dividing cells in vitro and in vivo (27).

The use of retroviral vectors for gene therapy has received much attention and currently is the method of choice for the transferral of therapeutic genes in a variety of approved protocols both in the USA and in Europe. However, most of these protocols require that the infection of target cells with the retroviral vector carrying the therapeutic gene occurs in vitro, and successfully infected cells are then returned to the affected individual. Such ex vivo gene therapy protocols are ideal for correction of medical conditions in which the target cell population can be easily isolated (e.g. lymphocytes). Additionally the ex vivo infection of target cells allows the administration of large quantities of concentrated virus which can be rigorously safety tested before use.

Unfortunately, only a fraction of the possible applications for gene therapy involve target cells that can be easily isolated, cultured and then reintroduced. Additionally, the complex technology and associated high costs of ex vivo gene therapy effectively preclude its disseminated use world-wide. Future facile and cost-effective gene therapy will require an in vivo approach in which the viral vector, or cells producing the viral vector, are directly administered to the patient in the form of an injection or simple implantation of retroviral vector producing cells. This is of course especially true for the cells of the central nervous system.

In vivo approaches, of course, introduce a variety of new problems. First of all, and above all, safety considerations have to be addressed. Virus will be produced, possibly from an implantation or injection of virus producing cells, and there will be no opportunity to precheck the produced virus. It is important to be aware of the finite risk involved in the use of such systems, as well as trying to produce new systems that minimize this risk.

Retroviral vector systems consist of two components:

1) the retroviral vector which itself is a modified retrovirus (vector plasmid) in which the genes encoding for the viral proteins have been replaced by therapeutic genes and marker genes to be transferred to the target cell. Since the replacement of the genes encoding for the viral proteins effectively cripples the virus it must be rescued by the second component in the system which provides the missing viral proteins to the modified retrovirus.

The second component is:

2) a cell line that produces large quantities of the viral proteins, however lacks the ability to produce replication competent virus. This cell line is known as the packaging cell line and consists of a cell line transfected with one or more plasmids carrying the genes enabling the modified retroviral vector to be packaged.

To generate the packaged vector, the vector plasmid is transfected into the packaging cell line. Under these conditions the modified retroviral genome including the inserted therapeutic and marker genes is transcribed from the vector plasmid and packaged into the modified retroviral particles (recombinant viral particles). This recombinant virus is then used to infect target cells in which the vector genome and any carried marker or therapeutic genes becomes integrated into the target cell's DNA. A cell infected with such a recombinant viral particle cannot produce new vector virus since no viral proteins are present in these cells. However the DNA of the vector carrying the therapeutic and marker genes is integrated in the cell's DNA and can now be expressed in the infected cell. Retroviruses are, as described above, constructed to minimize the chance of replication competent virus being present. However it is well documented that recombination events between components of the retrovirus vector/packaging system in fact can lead to the generation of potentially pathogenic and replication competent virus.

An additional complication with retroviral vectors is that only a few, all belonging to the lentivirus family, are known to be able to infect non-dividing cells. This includes Simian Immunodeficiency virus (SIV) as well as the Human Immunodeficiency Virus (HIV). It is clear that there will be severe difficulties in accepting HIV or SIV derived vectors, taking into account the very small, however real possibility of recombination events which may lead to pathogenic and replication competent HIV or SIV.

Gene therapy of central nervous system diseases and disorders will be completely dependent upon in vivo protocols.

### ABSTRACT OF THE INVENTION

The present invention addresses problems connected with gene therapy of non-dividing cells and especially the problems of developing gene therapy protocols suited for the application of gene therapy to treat central nervous system disorders and diseases. In contrast to oncoviruses, lentiviruses do not require target cell division for integration into the host genome. Therefore, lentiviral vectors could expand the spectrum of target cells susceptable to retroviral gene transfer. According to the present invention lentivirus based vectors for the use for gene transfer into non-dividing cells, comprising a vector/packaging system has been developed, in which Gag-Pol and the vector itself are of lentivirus origin, i.e. selected from the group consisting of HIV, human immunodeficiency virus type 1 and 2; SIV, simian immunodeficiency virus; FIV, feline immunodeficiency virus; BIV, bovine immunodeficiency virus; Visna/maedi virus; CAEV, caprine arthritis-encephalitis virus; or EIAV, Equine infectious anemia virus, while Env is derived either from one of above lentiviruses; or from mammalian C-type retroviruses like, amphotropic, polytropic or xenotropic murine leukemia viruses (MLV), murine sarcoma virus, feline leukemia viruses, simian sarcoma viruses, reticuloendotheliosis virus, or spleen necrosis virus; or from env of Rous sarcoma viruses; or from gibbon ape leukemia viruses; or from Spleen Nekrosis viruses; or from B-type viruses like mouse mammary tumor viruses; or from D-type viruses like Mason Pfizer monkey virus or Simian Retroviruses; or from HTLV, human T cell leukemia virus type 1 and 2; env of Spumaviruses like, Simiam foamy virus, Human foamy virus, or feline syncytium-forming virus; or from G-protein of vesicular stomatitis virus (VSV). Such lentivirus vectors pseudotyped with MLV or VSV-G-glycoprotein, for example, have titers of more than 10⁶ infectious units/ml. Growth arrested cells have been shown, according to the present invention, to be transduced efficiently with the SIV based vectors according to the present invention even if vpr, vpx, nef and vif have been mutated or deleted, and the nuclear localization signal or the C-terminal tyrosine of the matrix protein has been mutated. Therefore, it has surprisingly been found by the present invention that by the deletion of one or more or all of the vif, vpx, vpr, and nef genes from packaging and vector constructs according to the present invention it is still possible to obtain retroviral lentivirus vectors which both will efficiently infect and confer efficient gene transfer to non-dividing cells, while at the same time addressing the safety concerns with such vectors, obtained by the deletion of essential genes, thereby excluding the possibility that recombination events may occur which would make the virus again pathogenic, but not impairing transduction of non-dividing cells. The development of safe lentivirus based vectors thereby is possible.

For the treatment of central nervous system diseases or disorders any relevant therapeutic gene may be inserted into the viral vector of the present invention, this of course especially includes genes such as the NGF (nerve growth factor) gene, the GDNF (glia derived neurotrophic factor) gene, the DAT (dopamine transporter) gene, and the tyrosine hydroxylase gene, for example. As will be apparent to any person with an average knowledge in the field, any gene coding for a relevant protein may conveniently be inserted into the vector of the present invention. Likewise any therapeutic vector construct for the treatment of central nervous system diseases and disorders may be combined by and with the use in the vector construct of any promoter which may lead to gene expression in the relevant tissue. This of course also especially includes promoters specific for the central nervous system or any relevant subset of cells therof as well as any inducible promoter.

The vector/packaging system of the present invention may have utility outside the central nervous system. One example would be for example, metabolic liver diseases. In this case any person of an average skill in the art will immediately realise which therapeutic genes as well as which promoters will be relevant therefore.

It is of course clear that the vector/packaging system of the present invention will also infect dividing cells and therefore that the vector/packaging system of the invention may be combined with any suitable therapeutic gene, marker gene and promoter for use in dividing cells as well as therapeutic treatment of diseases or disorders of such cells.

In a further aspect the vector/packaging system of the present invention may, much like a replication deficient live vaccine, be used for the vaccination against lentivirus infection or the therapeutic vaccination of lentivirus infections and diseases; the immunogenic spectrum being dependent, of course, upon the exact genetic composition of the genes of the vector/packaging system of the present invention.

Pharmaceutical compositions and preparations comprising a vector/packaging system according to the present invention may be prepared according to standard and well known methods in the art for this purposes. All such methods are well known in the art and will be realised as such by any person of an average skill in the art.

After introducing the retroviral vector of the invention as described above in a retroviral packaging cell line a retroviral particle is provided comprising the recombinant retroviral genome. The invention also includes a retroviral provirus, mRNA of a retroviral provirus according to the invention, any RNA resulting from a retroviral vector according to the invention and cDNA thereof, as well as host cells infected with a retroviral particle according to the invention.

A further embodiment of the invention provides a method for introducing homologous and/or heterologous nucleotide sequences into target cells comprising infecting a target cell population in vivo and in vitro with recombinant retroviral particles.

The retroviral vector, the retroviral vector system and the retroviral provirus as well as RNA thereof is used for producing a pharmaceutical composition for in vivo and in vitro gene therapy in mammals including humans. Furthermore, they are used for targeted integration in homologous cellular sequences.

### SUMMARY OF THE INVENTION

The present invention then comprises the following, alone or in combination:

A lentivirus based vector comprising all or parts of the left and right hand LTR sequences, wherein the gag, pol and env coding sequences have all been partially or fully deleted or mutated and wherein one or more or all of the sequences coding for vif, vpr, vpx, and nef have independently or in combination wholly or partially been deleted, but where optionally the tat and rev genes are still expressed, and wherein the nuclear localisation signal and/or the C-terminal coding sequence of the matrix protein have optionally been deleted or mutated;

the retroviral lentivirus vector as above, comprising a gene relevant for the treatment of a central nervous system disease or disorder, including such genes such as the NGF (nerve growth factor) gene, the GDNF (glia derived neurotrophic factor) gene, the DAT (dopamine transporter) gene, or the tyrosine hydroxylase gene; or a gene relevant for metabolic liver disease or any other relevant disease;

a retroviral lentivirus based vector system comprising the lentivirus vector as above as a first component, and a packaging cell line that synthesises the Gag and Pol proteins of said lentivirus as well as the Env protein of the said lentivirus or of a heterologous Env protein, and where optionally the tat and rev genes are also expressed;

the retroviral lentivirus based vector system as above, wherein the vector is derived from HIV type 1 or 2, SIV, FIV, BIV, CAEV, EIAV, while Env is derived from mammalian C-type retroviruses like, amphotropic, polytropic or xenotropic murine leukemia viruses (MLV), murine sarcoma virus, feline leukemia viruses, simian sarcoma viruses, reticuloendotheliosis virus, or spleen necrosis virus; or from Rous sarcoma viruses; or from gibbon ape leukemia viruses; or from Spleen Nekrosis viruses; or from HIV, human immunodeficiency virus 1 and 2; or from SIV, simian immunodeficiency virus; or from B-type viruses like mouse mammary tumor viruses; or from D-type viruses like Mason Pfizer monkey virus or Simian Retroviruses; or from HTLV, human T cell leukemia virus type 1 and 2; or from Spumaviruses like, Simiam foamy virus, Human foamy virus, or feline syncytium-forming virus; or from G-protein of vesicular stomatitis virus (VSV-G);

the retroviral lentivirus vector system as above, wherein the vector is derived from SIV and the Env is derived from SIV or an amphotropic, polytropic or xenotropic murine leukemia virus or from vesicular stomatitis virus (VSV-G-protein);

a retroviral particle comprising a retroviral lentivirus based vector as above;

the retroviral particle as above obtainable by transfecting a packaging cell of the lentivirus based vector system as above with the lentivirus based vector as above;

a retroviral provirus produced by infection of target cells with the retroviral particle as above;

mRNA of a retroviral provirus as above;

RNA of the retroviral lentivirus based vector as above;

cDNA of the RNA as above;

a host cell infected with the retroviral particle as above;

the retroviral particle as above and/or the lentivirus based vector system as above and/or the lentivirus based vector as above for use in the treatment of a central nervous system disease or disorder or a metabolic liver disease or any other relevant disease or disorder;

a pharmaceutical composition containing a therapeutically effective amount of the retroviral particle as above and/or the retroviral lentivirus based vector system as above;

use of the lentivirus vector as above and/or of the retroviral lentivirus based vector system as above and/or of the retroviral particle as above for producing a pharmaceutical composition for gene therapy;

the use as above for the treatment of a central nervous system disease or disorder or a metabolic liver disease or any other relevant disease or disorder;

a method for introducing homologous and/or heterologous nucleotide sequences into target cells comprising infecting the target cells with retroviral particles as above;

a method of treating a central nervous system disorder or disease or metabolic liver disease or any other relevant disease or disorder of an animal including a human, which method comprises administering to a person in need thereof a therapeutically effective amount of the retroviral vector system as above and/or of the retroviral particle as above;

a method of immunising, by vaccination or therapeutic vaccination, an animal including a human, against lentivirus infection, which method comprises administering to a person in need thereof a therapeutically effective amount of the retroviral vector system as above and/or of the retroviral particle as above;

the method as above wherein the lentivirus infection is HIV or SIV or HTLV.

### EXAMPLES

Transfection and Infection

293T cells (293ts/A1609) (6) were obtained from ATCC and transfected with the calcium phosphate coprecipitation method as described (33). Transfection efficiency was determined by measuring the reverse transcriptase acitivity in the supernatant of transfected cells as described previously (18,29). CEMx174 cells were cultured in RPMI 1640 supplemented with 10% fetal calf serum, penicillin, streptomycin and glutamine. To block cell division, CEMx174 cells were γ-irradiated with 4000 rad.

1×10⁵ CEMx174 cells or 3×10⁵ irradiated CEMx174 cells were incubated for two hours with 150 μl and 450 μl vector supernatant, respectively. Cells were cultured for additional 48 hours after adding 1 ml medium. Luciferase activity was determined with the luciferase assay system (Promega, Madison, Wis.) as described by the manufacturer. The protein concentration of the cell extracts was determined by the Bio-Rad (München, Germany) protein assay. The titer of the SIV vectors was determined by infecting sMAGI cells (4) with serial dilutions of a vector preparation and counting the number of GFP positive cells two days after infection. For vector preparations containing the MLV Env 8 μg/ml polybrene (Sigma-Aldrich Chemie, Deisenhofen, Germany) was added. The MLV vector titer was determined on NIH3T3 cells using the vector pHIT111 as described (33). Human mononuclear cells were isolated from buffy coats by Ficoll-hypaque density centrifugation. 6 to 9×10⁶ cells were cultured per well of a six well plate in 3 ml DMEM medium supplemented with 10% fetal calf serum, 10% human AB serum, 350 μg glutamine/ml, 50 U GM-CSF/ml (Boehringer Mannheim) and antibiotics. Three to four days after seeding, wells were washed extensively with cold PBS and culture was continued. Every three to four days, non-adherent cells were removed and fresh medium was added. Two weeks after isolation, the adherent cells were incubated with 1 ml vector supernatant for three hours and culture was continued for 48 hours after addition of 3 ml medium. Microglia cells were isolated from thoroughly perfused brain tissue of uninfected and SIV infected rhesus monkeys using a percoll gradient technique and cultured as described (Sopper, S. et al. 1996: The effect of simian immunodeficiency virus infection in vitro and in vivo on the cytokine production of isolated microglia and periphal macrophages from rhesus monkey. Virology 220, 320). Isolated cells, which were >95% CD11b+, were transduced seven to ten days after isolation. Neurons were prepared from dorsal root ganglia of rats and cultured on coverslips for two days as described (Zeilhofer, H. U. et al. 1997: Fractional Ca²⁺ currents through capsaicin- and proton-activated ion channels in rat dorsal ganglion neurons. J. Physiol. 503, 67). Single coverslips were then exposed to 0.2 ml of the SIV vector preparation for three hours. After adding 0.8 ml medium, culture was continued for two days until immunofluorescence analysis.

Plasmids

S-gp: A deletion in env from 6603 to 7758 (numbering according to reference 30) was introduced as described previously (31) into SIVΔNU, a proviral clone of SIVmac239 containing deletions in nef and the U3 region (18). The 3′ LTR of this plasmid, designated SIVΔenvΔNU, was then replaced by a PCR generated fragment, which contained the MLV LTR with its polyadenylation site, resulting in S-gp.

S-env: A fragment comprising nucleotides 6706 to 10536 of the SIVmac239 provirus (GenBank entry: M33262) and cellular flanking regions was cloned into the HiidIII-EcoRI restriction site of pCDNAI-Amp (Invitrogen) resulting in S-env.

V1 and Vgp vectors: The coding region of the luciferase reporter gene was amplified by PCR and cloned in place of nef into a unique Xma I restriction site of SIVΔenvΔNU resulting in Vgp-luc. Expression of SIV gng-pol was blocked by introducing two stop codons at codon eight and nine of gng. In addition, the vif gene was replaced by a vif gene containing a large deletion (17), which was kindly provided by R. C. Desrosiers through the AIDS Research and Reference Reagent Program resulting in V1-luc. The luciferase gene of V1-luc and Vgp-luc was replaced by the E-GFP gene (Clontech), which had been amplified by PCR, resulting in V1-gfp and Vgp-gfp, respectively. To construct Vgp-Prgfp, the luciferase gene of Vgp-luc was replaced by an expression cassette consisting of the promoter region of spleen focus forming virus (Baum, C. et al. 1995: Novel retroviral vectors for efficient expression of the multidrug resistance (mdr-1) gene in early hematopoietic cells. J. Virol. 69, 7541) and the E-GFP gene.

Vgp-luc derivatives: In R− the vpr start codon was mutated to TTG (24). In X− the start codon of vpx was mutated to ACG and the second codon was changed to the stop codon TAA. Neither of the vpx mutations altered the overlapping vif reading frame. A putative nuclear localization signal (NLS) within the matrix protein of SIVmac239, which is highly conserved among HIV-1 and SIV, was mutated from ²⁵GKKKYMLK to ²⁵GTTKYMLK (nucleotide sequence: ggaaccactaagtacatgttgaag). The resulting plasmid was designated N−. To prevent phosphorylation of the C-terminal tyrosine of the matrix protein, this amino acid was mutated to phenylalanine, resulting in Y−. These mutations were also cloned in various combinations into Vgp-luc resulting in RX−, RY−, RN−, XY−, XN−, RXY−, RXN−. In Δfrx, a large deletion spanning the vif, vpr, and vpx genes (described in reference 17; kindly provided by R. C. Desrosiers through the AIDS Research and Reference Reagent Program) was introduced into Vgp-luc.

MLV and VSV plasmids: The plasmids pHIT60 (MLV gng-pol expression plasmid, here referred to as M-gp), pHIT456 (amphotropic MLV env expression similar to pHIT123, here referred to as M-env), pHIT111 (MLV β-galactosidase vector) and pHIT-G (VSV-G expression plasmid) are described by Soneoka et al. (33) and Fouchier, R. A. M. et al. (1997: HIV-1 infection of non-dividing cells: evidence that the amino-terminal basic region of the viral matrix protein is important for Gag processing but not for post-entry nuclear import; EMBO 16, 4531). Plasmid pRV172 is a pHIT version of pLNCX (Genbank accession number: M28247), in which the luciferase gene was inserted under the control of the CMV-IE promoter, and was provided by P. Cannon.

Flow Cytometry

Monocyte derived macrophages were stained with a 1:10 dilution of the anti-CD11c antibody (Leu M5) labelled with phycoerythrin (Becton Dickinson) or an isotype matched control according to standard procedures. For the detection of GFP, cells were fixed for 10 min in 150 μl 1% paraformaldehyde at 4° C. Cells were analyzed by flow cytometry using a FACStract analyzer with Lysis II software (Becton Dickinson). For cell cycle analysis, cellular DNA was stained with propidium iodine as described (Hofman, F. 1994: Flow cytometry, in: Current protocolls in immunology, 2nd Edition; Eds: Coligan, J. E. et al., John Wiley & Sons, Inc, USA). Flow cytometry was then performed with a Coulter Epics Elite analyzer with Multicycle software (Phoenix).

Transient Three Plasmid Vector/Packaging System

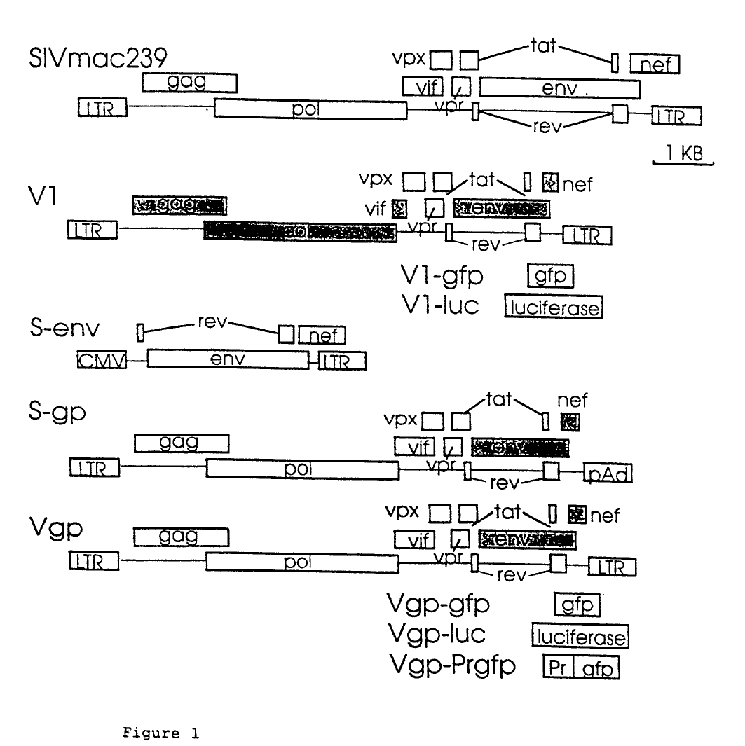

To generate SIV based vectors, a transient three plasmid vector/packaging system was used. Since SIV sequences required in cis were not well defined, at first small deletions and mutations were introduced into the SIV vector to inactivate viral genes without altering the genomic organization of the virus. A map of the vector V1 is shown in FIG. 1. Since a mutation close to the start codon of gng of HIV-1 reduced packaging of the viral RNA (25), two stop codons were introduced at amino acid 8 and 9 of gng of V1 instead of mutating the start codon. Since no reverse transcriptase activity could be detected after transfection of plasmids containing these mutations, gng-pol expression was blocked as expected. V1 also contains a deletion in vif and a deletion of the first 1154 bp of the env gene. The deletion in env did not affect the Rev-RRE regulation as indicated by efficient replication of a hybrid virus containing this deletion (31). In addition, a deletion of 513 bp in nef and the U3 region was introduced, which have been described previously (18). To be able to detect transfer of VI, the gene of the green fluorescence protein (GFP) or the luciferase gene was inserted in place of nef resulting in V1-gfp or V1-luc, respectively. To package V1-gfp, an SIV env expression plasmid (S-env) and an SIV gng-pol expression plasmid (S-gp) were constructed, both of which also express some of the regulatory genes (FIG. 1). Cotransfection of V1-gfp, S-gp and S-env into 293T cells resulted in retroviral vector particles, containing an SIV core and the SIV envelope proteins (SIV[SIV]). The vector titer was determined on sMAGI cells (4) with the help of the GFP reporter gene. A titer of about 1×10⁵ infectious units/ml was obtained (Table 1). To analyze whether SIV vector particles could be pseudotyped efficiently, an expression plasmid for the env gene of amphotropic MLV (M-env) or the G-protein of vesicular stomatitis virus (VSV-G) were cotransfected with S-gp and V1-gfp resulting in SIV[MLV] or SIV[VSV] vector particles. Titers of up to 5×10⁶/ml were obtained (Table 1). In the absence of either the env expression plasmid or the SIV gng-pol expression plasmid, titers were reduced to background levels (Table 1). In the presence of 10 μM of the reverse transcriptase inhibitor Zidovudine (3′-azido-2′,3′-dideoxythymidine; AZT), the transfer of V1-luc by SIV[SIV] or SIV[MLV] vector particles into CEMx174 cells was inhibited by 90%. This confirms that the observed gene transfer is indeed retrovirally mediated and not a passive transfer of vector DNA.

Transduction of Non-dividing Cells

To analyze whether non-dividing cells can be transduced, CEMx174 cells were irradiated with 4000 rad. At 1, 2, and 3 days after irradiation, H³-thymidine incorporation was reduced to background levels. 24 hours after irradiation, irradiated and non-irradiated cells were infected at the same multiplicity with different vector particles transferring the luciferase gene. The specific luciferase activity was determined 2 days later. The ratio of the specific luciferase activities of irradiated to non-irradiated cultures infected with the same supernatant was taken as the transduction efficiency of growth arrested cells. For SIV[SIV] vector particles a transduction efficiency of 0,29 was obtained (Table 2). Using SIV[MLV] vector particles, the transduction efficiency of growth arrested cells was reduced by a factor of approximately 3 in three independent experiments, which has been performed in triplicate. The SIV env expression plasmid might also allow expression of nef (see FIG. 1). To exclude that the higher transduction efficiencies of irradiated cells in the presence of the SIVenv expression plasmid is in fact due to nef expression, the same experiment was performed with a modified SIV env expression plasmid, in which the nef gene had been deleted. Again, the ratio of the luciferase activities of irradiated to non-irradiated cells infected with the modified SIV[SIV] vector was approximately threefold higher than for the SIV[MLV] vector. The reduced transduction efficiencies of irradiated cells by SIV[MLV] vectors were partially compensated by higher titers, indicating efficient infection of non-dividing cells in the absence of the SIV env gene. Since more than 50% of cells were dead three days after irradiation, the difference in transduction efficiency of irradiated and non-irradiated cells by SIV based vectors should be even smaller. Using a MLV based vector, luciferase activities were much lower in non-irradiated CEMx174 cells (Table 2). This could be due to inefficient transduction of human lymphoid cells by MLV vectors. Alternatively, the transcriptional activity of the CMV-promoter, which regulates expression of the luciferase reporter gene of the MLV vector, might be reduced in comparison to the SIV-LTR. Nevertheless, since no luciferase activity could be detected at all in irradiated cells, the ratio of the luciferase activities of irradiated to non-irradiated cells infected with the MLV vector was significantly lower than the corresponding ratio of SIV based vectors. This was due to a stronger reduction of the transcriptional activity of the promoter driving the expression of the luciferase gene of the MLV vector, since the ratios of the luciferase activities of irradiated and non-irradiated CEMx174 cells, which had been infected with the SIV luciferase vector or the MLV luciferase vector 24 hours prior to irradiation, were similar. Therefore, SIV and MLV vectors clearly differ in their transduction efficiency of non-dividing cells.

Transduction of Primary Human Cells

To determine whether cells arrested in the cell cycle in a more natural way could be transduced by SIV based vectors, human foreskin fibroblasts were grown to confluence for two weeks. This led to a reduction in ³H thymidine incorporation by 87% (Table 3). Fibroblasts plated 2, 7, or 14 days before were exposed to SIV and MLV vectors transferring the luciferase gene, and luciferase activity was determined 2 days later. SIV based vectors were a factor of three to five less sensitive to the growth arrest of fibroblasts than MLV based vectors (Table 3).

Terminally differentiated human macrophage cultures were also exposed to V1-gfp and the GFP expressing SIV vector Vgp-gfp both pseudotyped with VSV-G or M-env. Vgp-gfp is similar to V1-gfp but still allows expression of gng-pol. The titers obtained with Vgp-gfp were higher than with V1-gfp. FACS analyses two days after infection revealed that after pseudotyping with VSV-G up to 35% of the cells had been transduced with Vgp-gfp and 10% with V1-gfp. 99% of the transduced cells also expressed the monocyte/macrophage marker CD11c. Since more than 85% of the cells were in Gₒ/Gₗ phase of the cell cycle at the time of infection, non-dividing macrophages must have been transduced by the SIV vector. In contrast to VSV-G pseudotyped SIV vectors, MLV Env pseudotyped vectors were very inefficient in transducing macrophages.

Transduction of Cells of the Nervous System

Microglia cells are potential targets for gene therapy of disorders of the central nervous system. Microglia cells were prepared from the brain of rhesus monkeys. About 90% of all microglial cells derived from the brain of rhesus monkeys were in the Gₒ/Gₗ phase of the cell cycle. Despite that, 75% of the cells could be transduced with the VSV-G pseudotyped Vgp-gfp. Transduction with MLV Env pseudotypes of Vgp-gfp was possible but less efficient. Other potential target cells of the nervous system are neurons, which usually do not divide in adults at all. To analyze whether they could be transduced, neuron cultures from the dorsal root ganglia of rats were exposed to the SIV vector Vgp-Prgfp (FIG. 1) pseudotyped with VSV-G. To allow efficient GFP expression in rodents, an internal promoter had been inserted upstream of the GFP gene. Immunofluorescence microscopy revealed that cells with typical neuron like morphology were efficiently transduced with the VSV-G pseudotyped SIV vector. Cells with this morphology have membrane conductance properties which are only found in neurons.

Requirements for Infection of Non-dividing Cells

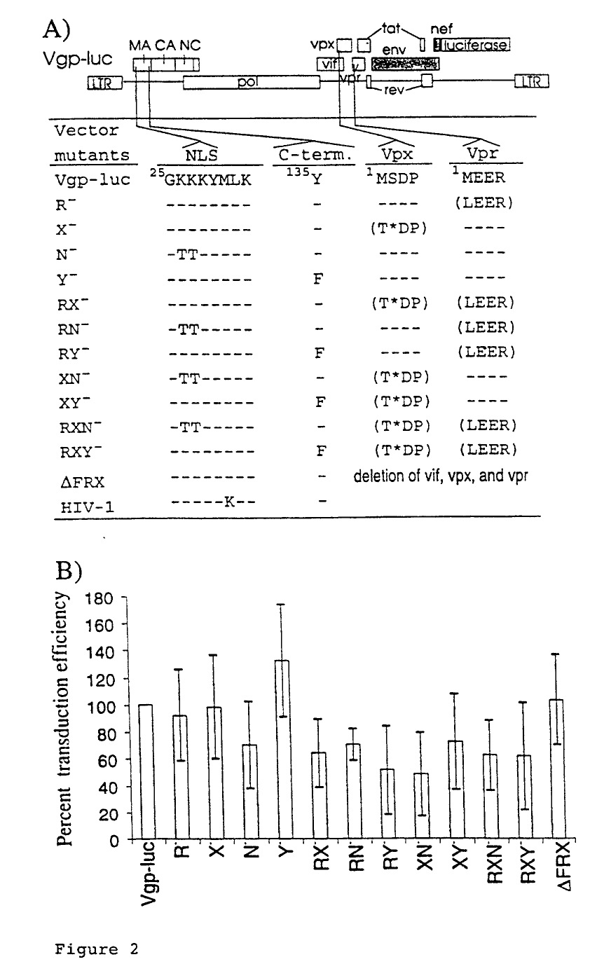

Infection of non-dividing cells by HIV-1 was reported to depend in a partially redundant way on the accessory protein Vpr and a nuclear localization signal (NLS) of the matrix protein (3,19,34). To analyze the genetic requirements of SIV for infection of non-dividing cells, a set of mutants of the SIV vector Vgp-luc were constructed (FIG. 2A). The same mutations that inactivate the NLS of HIV-1 matrix protein were introduced into the highly conserved region of the SIV matrix protein. Since it was reported that the nuclear transport function of the NLS of HIV-1 matrix protein and the infectivity for non-dividing cells could also be abrogated by mutating the C-terminal tyrosine (13,14), this mutation was also introduced into the SIV vector. To prevent vpr expression the start codon of vpr (24) was mutated. In addition to vpr, most SIV strains contain a vpr related gene, vpx, which is important for infection of monocyte-derived macrophages (8). Since vpx could also play a more general role in the infection of non-dividing cells, the vpx reading frame was also inactivated. Each of these mutations were introduced alone or in various combinations into Vgp-luc (FIG. 2A). After cotransfection of Vgp-luc mutants with the MLV enz expression plasmid, non-irradiated and irradiated CEMx174 cells were infected and the transduction efficiency was determined. As shown in FIG. 2B, neither of the single mutations nor any of the double or triple mutations severely affected the transduction efficiency of irradiated cells. To determine the role of vif for the transduction of non-dividing cells, the vif, vpr and vpx genes were deleted from Vgp-luc resulting in Δfrx. Growth arrested cells were transduced with Δfrx as efficiently as with Vgp-luc (FIG. 2B).

To further analyze viral requirements for infection of non-dividing cells, Vgp-luc and the vif, vpr and vpx deletion mutant of Vgp-luc, ΔFRX, were pseudotyped with VSV-G. Terminally differentiated macrophages and rapidly dividing CEMx174 cells were infected with both vector stocks. The luciferase activity/μg cell extract was measured and the ratio of the specific luciferase activities of macrophages to CEMx174 cells was calculated for both vectors. The ratios of the luciferase activities of the two vectors did not differ significantly demonstrating that efficient transfer into non-dividing macrophages did not depend on the vif, vpr, or vpx genes. Nef was not required either, since it is deleted in Vgp-luc and AFRX.

Discussion

There seem to be at least two main obstacles to the use of retroviral vectors for in vivo gene delivery. A problem for the use of retroviral vectors for in vivo gene delivery is that cell division is required for transduction with conventional retroviral vectors (26,32). Immunodeficiency viruses can infect non-dividing cells (15,20,35). Immunodeficiency virus based vectors could therefore greatly expand the range of potential target cells for retroviral gene transfer.

Vectors based on immunodeficiency viruses harbor a number of risks due to the pathogenicity of the parental viruses. Clearly, the emergence of replication-competent recombinants (RCR) between the vector and the packaging construct has to be prevented. But this is also a prerequist for safe MLV vectors, since RCRs of MLV vectors were found to induce leukemia in immunosuppressed non human primates (5). Since MLV vector/packaging systems, in which no RCRs are detected, have already been developed, this should also be achievable for immunodeficiency virus based vectors. However, in addition to lack of RCR, which can be demonstrated in vitro, it will also be necessary to evaluate the safety of immunodeficiency virus based vectors in a relevant animal model prior to use in humans. Since HIV-1 only causes AIDS in humans, no good animal model exists to evaluate the risk associated with HIV based vectors.

Therefore, an SIV based vector has been developed. Although pathogenicity of SIV for humans can not be excluded, the course of laboratory acquired SIV infections suggests greatly reduced virulence in comparison to HIV (23). More importantly, since SIV induces AIDS in rhesus monkeys, SIV vectors that are safe in macaques should also be safe in humans.

The results demonstrate, that SIV based vectors can transduce a variety of non-dividing cells when pseudotyped appropriately. The titers obtained were in a similar range as MLV based vectors. The pathogenicity of SIV not only depends on functional gng, pol and env genes, but also on accessory genes. By removing these accessory genes from SIV based vectors and packaging constructs, the emergence of wild type virus can be excluded. According to the present invention it has been found that it is feasible to construct lentivirus retroviral based vectors, which have had one or more or all of their vif, vpr, vpx, and nef deleted without loosing their capacity to transduce non-dividing cells. It has also been observed, that growth arrested cells could be transduced even if vpr, vpx, and the NLS or the C-terminal tyrosine of the matrix protein had been mutated. Although it was initially reported that mutating the NLS of the matrix protein and inactivating vpr of HIV-1 greatly reduced infection of non-dividing cells (3,13,14,19,34), more recent results suggest that at least one additional NLS bearing protein might play a role in the import of the preintegration complex into the nucleus of non-dividing cells (12). Since HIV-1 was also found to replicate to high titers in terminally differentiated macrophages even if the NLS or the C-terminal tyrosine of MA and Vpr were mutated (9,10), some other factor must indeed mediate the transport of the preintegration complex into the nucleus of non-dividing cells. The results indicate that for SIV this function seems to be retained in the gng, pol, tat or rev gene, since all other reading frames could be inactivated in the packaging constructs and the vector without loss of infectivity for non-dividing CEMx174 cells.

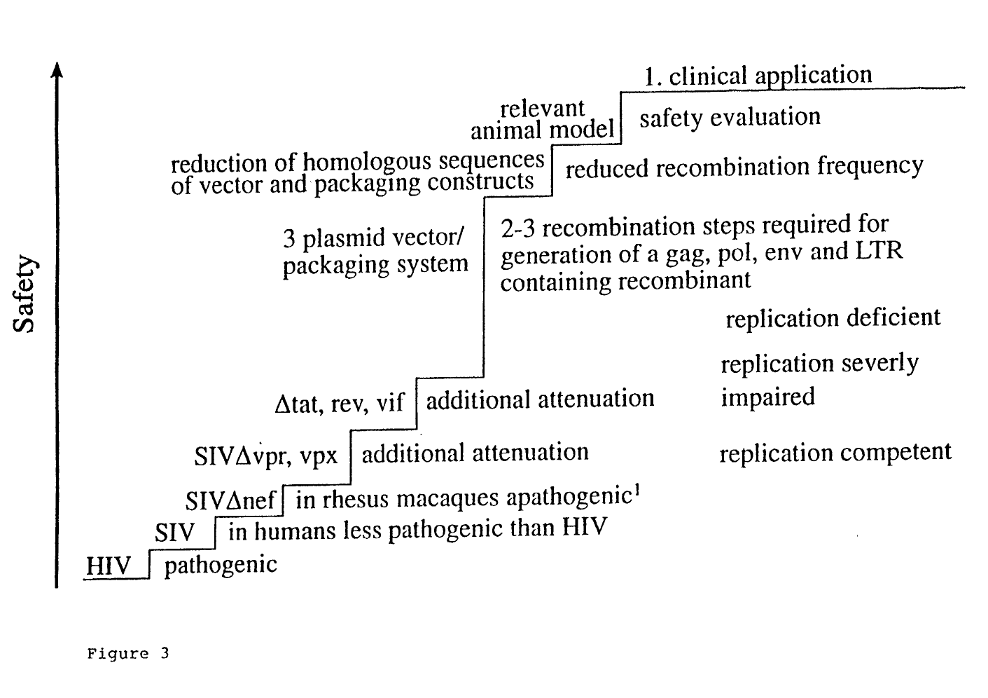

All accessory genes, that are not required for transduction of non-dividing cells, can be deleted from SIV vector/packaging systems. Each of these genes contribute to the pathogenicity of SIV. Nef deletion mutants of SIV are apathogenic (22) unless injected in high doses in neonates (1,36). Deletion of vpr and/or vpx seems to prevent or delay the onset of SIV induced disease (16,21). In the absence of Vif, replication of SIV is severly impaired (17,28,40). Since Vif is only required at the time of virus production in some cells (7,11), it was possible to delete vif by choosing an appropriate vector producer cell line. In addition to deletion of accessory genes, the use of heterologous env genes might also contribute to the safety of SIV based vectors. By eliminating homologous regions between vector and packaging constructs, the recombination frequency can be reduced. Since a well characterized animal model is available for SIV based vectors, it is also possible to analyze the worst case scenario, the emergence of a RCR, by reconstructing potential recombinants between SIV and the heterologous env genes (31). The different safety levels, which can be incorporated into SIV based vectors (summarized in FIG. 3), might allow the development of a save retroviral gene transfer method for non-dividing cells, which could be used for in vivo gene therapy.

The vector/packaging system of the present invention therefore provides a safe lentivirus based vector system which efficiently will deliver genes into non-dividing cells.

The invention may be worked according to numerous equivalent or similar procedures all being well known in the art. Especially the packaging/vector construct necessary to work the invention may be synthesized according to different strategies and the viral genes necessary to assemble the recombinant viral particle may come from the same or different plasmids of the packaging cell for example. All of such equivalent or similar procedures to obtain and effect the steps of the present invention, will be appreciated as such by any person of average skill in the art, and should be considered part of and comprised by the present invention and application and the invention is therefore only to be limited by the full scope of the appended claims.

References

1. Baba, T. W., Y. S. Jeong, D. Pennick, R. Bronson, M. F. Greene, and R. M. Ruprecht. 1995. Pathogenicity of live, attenuated SIV after mucosal infection of neonatal macaques. Science 267:1820-1825.

2. Brenner, M. K., J. M. Cunningham, B. P. Sorrentino, and H. E. Heslop. 1995. Gene transfer into human hemopoietic progenitor cells. Br. Med. Bull. 51:167-191.

3. Bukrinsky, M. I., S. Haggerty, M. P. Dempsey, N. Sharova, A. Adzhubel, L. Spitz, P. Lewis, D. Goldfarb, M. Emerman, and M. Stevenson. 1993. A nuclear localization signal within HIV-1 matrix protein that governs infection of non-dividing cells. Nature 365:666-669.

4. Chackerian, B., N. L. Haigwood, and J. Overbaugh. 1995. Characterization of a CD4-expressing macaque cell line that can detect virus after a single replication cycle and can be infected by diverse simian immunodeficiency virus isolates. Virology 213:386-394.

5. Donahue, R. E., S. W. Kessler, D. Bodine, K. McDonagh, C. Dunbar, S. Goodman, B. Agricola, E. Byrne, M. Raffeld, R. Moen, J. Bacher, K. M. Zsebo, and A. W. Nienhuis. 1992. Helper virus induced T cell lymphoma in nonhuman primates after retroviral mediated gene transfer. J. Exp. Med. 176:1125-1135.

6. DuBridge, R. B., P. Tang, H. C. Hsia, P. M. Leong, J. H. Miller, and M. P. Calos. 1987. Analysis of mutation in human cells by using an Epstein-Barr virus shuttle system. Mol. Cell Biol. 7:379-387.

7. Fan, L. and K. Peden. 1992. Cell-free transmission of Vif mutants of HIV-1. Virology 190:19-29.

8. Fletcher, T. M. I., B. Brichacek, N. Sharova, M. A. Newman, G. Stivahtis, P. M. Sharp, M. Emerman, B. H. Hahn, and M. Stevenson. 1997. Nuclear import and cell cycle arrest function of the HIV-1 Vpr protein are encoded by two separate genes in HIV-2/SIVsm. EMBO 15:6155-6165.

9. Freed, E. O., G. Englund, F. Maldarelli, and M. A. Martin. 1997. Phosphorylation of residue 131 of HIV-1 matrix is not required for macrophage infection. Cell 88:171-174.

10. Freed, E. O., G. Englund, and M. A. Martin. 1995. Role of the basic domain of human immunodeficiency virus type 1 matrix in macrophage infection. J. Virol. 69:3949-3954.

11. Gabuzda, D. H., K. Lawrence, E. Langhoff, E. Terwilliger, T. Dorfman, W. A. Haseltine, and J. Sodroski. 1992. Role of vif in replication of human immunodeficiency virus type 1 in CD4+ T lymphocytes. J. Virol. 66:6489-6495.

12. Gallay, P., V. Stitt, C. Mundy, M. Oettinger, and D. Trono. 1996. Role of the karyopherin pathway in human immunodeficiency virus type 1 nuclear import. J. Virol. 70:1027-1032.

13. Gallay, P., S. Swingler, C. Aiken, and D. Trono. 1995. HIV-1 infection of nondividing cells: C-terminal tyrosine phosphorylation of the viral matrix protein is a key regulator. Cell 80:379-388.

14. Gallay, P., S. Swingler, J. Song, F. Bushman, and D. Trono. 1995. HIV nuclear import is governed by the phosphotyrosine-mediated binding of matrix to the core domain of integrase. Cell 83:569-576.

15. Gartner, S., P. Markovits, D. M. Markovitz, M. H. Kaplan, R. C. Gallo, and M. Popovic. 1986. The role of mononuclear phagocytes in HTLV-III/LAV infection. Science 233:215-219.

16. Gibbs, J. S., A. A. Lackner, S. M. Lang, M. A. Simon, P. K. Sehgal, M. D. Daniel, and R. C. Desrosiers. 1995. Progression to AIDS in the absence of a gene for vpr or vpx. J. Virol. 69:2378-2383.

17. Gibbs, J. S., D. A. Regier, and R. C. Desrosiers. 1994. Construction and in vitro properties of SIVmac mutants with deletions in non essentiel genes. AIDS Res. Hum. Retroviruses 10:607-616.

18. Gundlach, B. R., H. Linhart, U. Dittmer, S. Sopper, S. Reiprich, D. Fuchs, B. Fleckenstein, G. Hunsmann, C. Stahl-Hennig, and K. gberla. 1997. Construction, Replication, and Immunogenic Properties of a Simian Immunodeficiency Virus expressing Interleukin 2. J. Virol. 71:2225-2232.

19. Heinzinger, N. K., M. I. Bukinsky, S. A. Haggerty, A. M. Ragland, V. Kewalramani, M. A. Lee, H. E. Gendelman, L. Ratner, M. Stevenson, and M. Emerman. 1994. The Vpr protein of human immunodeficiency virus type 1 influences nuclear localization of viral nucleic acids in nondividing host cells. Proc. Natl. Acad. Sci. U. S. A. 91:7311-7315.

20. Ho, D. D., T. R. Rota, and M. S. Hirsch. 1986. Infection of monocyte/macrophages by human T lymphotropic virus type III. J. Clin. Invest. 77:1712-1715.

21. Hoch, J., S. M. Lang, M. Weeger, C. Stahl Hennig, C. Coulibaly, U. Dittmer, G. Hunsmann, D. Fuchs, J. Mhller, S. Sopper, B. Fleckenstein, and K. gberla. 1995. vpr deletion mutant of simian immunodeficiency virus induces AIDS in rhesus monkeys. J. Virol. 69:4807-4813.

22. Kestler, H. W., D. J. Ringler, K. Mori, D. L. Panicali, P. K. Sehgal, M. D. Daniel, and R. C. Desrosiers. 1991. Importance of the nef gene for maintenance of high virus loads and for development of AIDS. Cell 65:651-662.

23. Khabbaz, R. F., W. Heneine, J. R. George, B. Parekh, T. Rowe, T. Woods, W. M. Switzer, H. M. McClure, M. Murphey Corb, and T. M. Folks. 1994. Brief report: infection of a laboratory worker with simian immunodeficiency virus. N. Engl J. Med. 330:172-177.

24. Lang, S. M., M. Weeger, C. Stahl Hennig, C. Coulibaly, G. Hunsmann, J. Mhller, H. K. Mhller-Hermelink, D. Fuchs, H. Wachter, M. M. Daniel, R. C. Desrosiers, and B. Fleckenstein. 1993. Importance of vpr for infection of rhesus monkeys with simian immunodeficiency virus. J. Virol. 67:902-912.

25. Luban, J. and S. P. Goff. 1994. Mutational analysis of cis-acting packaging signals in human immunodeficiency virus type 1 RNA. J. Virol. 68:3784-3793.

26. Miller, D. G., M. A. Adam, and A. D. Miller. 1990. Gene transfer by retrovirus vectors occurs only in cells that are actively replicating at the time of infection. Mol. Cell Biol. 10:4239-4242.

27. Naldini, L., U. Bl'mer, P. Gallay, D. Ory, R. Mulligan, F. H. Gage, I. M. Verma, and D. Trono. 1996. In vivo gene delivery and stable transduction of nondividing cells by a lentiviral vector. Science 272:263-267.

28. Park, I. W., K. Myrick, and J. Sodroski. 1994. Effects of vif mutations on cell-free infectivity and replication of simian immunodeficiency virus. J. Acquir. Immune. Defic. Syndr. 7:1228-1236.

29. Potts, B. J. 1990. “Mini reverse transcriptase (RT) assay, p. 103-106. In A. Aldovini and B. D. Walker (eds.), Techniques in HIV research. Stockton Press, New York.

30. Regier, D. A. and R. C. Desrosiers. 1990. The complete nucleotide sequence of a pathogenic molecular clone of simian immunodeficiencv virus. AIDS Res. Hum. Retroviruses 6:1221-1231.

31. Reiprich, S., R. B. Gundlach, B. Fleckenstein, and K. gberla. 1997. Replication competent chimeric Lenti-Oncovirus with expanded host cell tropsim. J. Virol. in press:

32. Roe, T., T. C. Reynolds, G. Yu, and P. O. Brown. 1993. Integration of murine leukemia virus DNA depends on mitosis. EMBO J. 12:2099-2108.

33. Soneoka, Y., P. M. Cannon, E. E. Ramsdale, J. C. Griffiths, G. Romano, S. M. Kingsman, and A. J. Kingsman. 1995. A transient three-plasmid expression system for the production of high titer retroviral vectors. Nucleic. Acids. Res. 23:628-633.

34. von Schwedler, U., R. S. Kornbluth, and D. Trono. 1994. The nuclear localization signal of the matrix protein of human immunodeficiency virus type I allows the establishment of infection in macrophages and quiescent T lymphocytes. Proc. Natl. Acad. Sci. U. S. A. 91:6992-6996.

35. Weinberg, J. B., T. J. Matthews, B. R. Cullen, and M. H. Malim. 1991. Productive human immunodeficiency virus type 1 (HIV-1) infection of nonproliferating human monocytes. J. Exp. Med. 174:1477-1482.

36. Wyand, M. S., K. H. Manson, A. A. Lackner, and R. C. Desrosiers. 1997. Resistance of neonatal monkeys to live attenuated vaccine strains of simian immunodeficiency virus. Nature Med. 3:32-36.

37. Yee, J. K., A. Miyanohara, P. LaPorte, K. Bouic, J. C. Burns, and T. Friedmann. 1994. A general method for the generation of high-titer, pantropic retroviral vectors: highly efficient infection of primary hepatocytes. Proc. Natl. Acad. Sci. U. S. A. 91:9564-9568.

38. Zack, J. A., S. J. Arrigo, S. R. Weitsman, A. S. Go, A. Haislip, and I. S. Chen. 1990. HIV-1 entry into quiescent primary lymphocytes: molecular analysis reveals a labile, latent viral structure. Cell 61:213-222.

39. Zack, J. A., A. M. Haislip, P. Krogstad, and I. S. Chen. 1992. Incompletely reverse-transcribed human immunodeficiency virus type 1 genomes in quiescent cells can function as intermediates in the retroviral life cycle. J. Virol. 66:1717-1725.

40. Zou, J. X. and P. A. Luciw. 1996. The requirement for Vif of SIVmac is cell-type dependent. J. Gen. Virol. 77:427-434.

TABLE 1
Comparison of vector titers

| Supernatant of 293T | titer (FFU^{a}/ml) | |
|---|---|---|
| cells transfected with | Exp. 1 | Exp. 2 |
| V1-gfp, S-gp, S-env | 1 × 10⁵ | 2.4 × 10³ |
| V1-gfp, S-gp, M-env | 3.8 × 10⁶ | 2.7 × 10⁶ |
| V1-gfp, S-gp, VSV-G | 5.2 × 10⁶ | 5.4 × 10⁶ |
| V1-gfp, S-gp | <6.6 | n.d. |
| V1-gfp, VSV-G | <6.6 | n.d. |

^{a}FFU: Fluorescence forming units.
V1-gfp: SIV vector;
S-gp: SIV gng-pol expression plasmid;
S-env: SIV env expression plasmid;
M-env: MLV env expression plasmid;
VSV-G: VSV-G expression plasmid.

TABLE 2
Transduction of growth-arrested CEM × 174 cells

| Supernatant of 293T | Luciferase activity^{a} | (RLU/μg^{b}) | Transduction |
|---|---|---|---|
| cells transfected with | non-irradiated | irradiated | efficiency |
| V1-luc, S-gp, S-env | 304 ± 27 | 89 ± 3 | 0.29 |
| V1-luc, S-gp, M-env | 473 ± 40 | 41 ± 3 | 0.09 |
| pRV172, S-gp, S-env | <0.125 | <0.125 | |
| pRV172, M-gp, M-env | 5 ± 0.7 | <0.125 | <0.025 |

^{a}mean of three independent experiments performed in triplicates;
^{b}realtive light units/μg cell extract;
V1-luc: SIV vector transfering the luciferase gene;
pRV172: MLV vector transfering the luciferase gene;
other plasmids are described in Table 1.

TABLE 3
Transduction of growth-arrested fibroblasts

| | | |
|---|---|---|
| Days in | % Luciferase activity^{a} | % ³H-thymidine |

| culture | SIV^{b} | MLV^{C} | incorporation |
|---|---|---|---|
| 2 | 100 | 100 | 100 |
| 7 | 95 ± 35 | 30 ± 15 | 41 ± 30 |
| 14 | 22 ± 10 | 4 ± 4 | 13 ± 7 |

^{a}The percentage of the mean luciferase activities of triplicates was determined 2 days after infection of cells that had been plated 2, 7 or 14 days before. The 2 day value was set as 100%. The mean of three independent experiments ± standard deviation is shown.
^{b}SIV vectors generated by cotransfection of V1-luc, S-gp, and M-env into 293T cells.
^{c}MLV vectors were generated by cotransfection of pRV172, M-gp, and M-env into 293T cells.

Figure Legends

FIG. 1. SIV packaging and vector constructs. Inactivated reading frames are marked by a shaded box; deletions are indicated by a vertical indented line within a shaded box. gfp: green fluorescence protein; pAd: heterologous polyadenylation signal derived from MLV; CMV: immediated early promoter/enhancer of human cytomegalovirus; Pr: heterologous promoter derived from spleen focus forming virus.

FIG. 2. Viral determinants of infectivity for non-dividing cells. A.) A map of the SIV based vector Vgp-luc is shown above the table of Vgp-luc mutants. Inactivated reading frames are marked by a shaded box. Numbers in the table give the amino acid number of the respective protein. Amino acids are given in the one letter symbol; asterisks indicate stop codons; MA: matrix protein; CA: capsid protein; NC: nucleocapsid protein; NLS: nuclear localization signal of the matrix protein; C-term.: carboxyterminal amino acid of the matrix protein. B. Percent transduction efficiency of non-dividing cells. The ratio of the specific luciferase activities in non-dividing and dividing cells is expressed as percent of the ratio obtained with Vgp-luc. The mean of triplicates±standard deviation is given.

FIG. 3. Possible safety levels of SIV based vectors. ¹except infection of neonatal rhesus monkeys at high doses (1,36).

1. A lentivirus based vector comprising all or parts of the left and right hand LTR sequences, wherein the gng, pol and env coding sequences have all been partially or fully deleted or mutated and wherein one or more or all of the sequences coding for vif, vpr, vpx, and nef have independently or in combination wholly or partially been deleted, but where optionally the tat and rev genes are still expressed, and wherein the nuclear localisation signal and/or the C-terminal coding sequence of the matrix protein have optionally been deleted or mutated.

2. The retroviral lentivirus vector according to claim 1, comprising a gene relevant for the treatment of a central nervous system disease or disorder, including such genes such as the NGF (nerve growth factor) gene, the GDNF (glia derived neurotrophic factor) gene, the DAT (dopamine transporter) gene, or the tyrosine hydroxylase gene; or a gene relevant for metabolic liver disease or any other relevant disease.

3. A retroviral lentivirus based vector system comprising the lentivirus vector according to claim 1 or 2 as a first component, and a packaging cell line that synthesises the Gag and Pol proteins of said lentivirus as well as the Env protein of the said lentivirus or of a heterologous Env protein, and where optionally the tat and rev genes are also expressed.

4. The retroviral lentivirus based vector system according to claim 3, wherein the vector is derived from HIV type 1 or 2, SIV, FIV, BIV, CAEV, EIAV, while Env is derived from mammalian C-type retroviruses like, amphotropic, polytropic or xenotropic murine leukemia viruses (MLV), murine sarcoma virus, feline leukemia viruses, simian sarcoma viruses, reticuloendotheliosis virus, or spleen necrosis virus; or from Rous sarcoma viruses; or from gibbon ape leukemia viruses; or from Spleen Nekrosis viruses; or from HIV, human immunodeficiency virus 1 and 2; or from SIV, simian immunodeficiency virus; or from B-type viruses like mouse mammary tumor viruses; or from D-type viruses like Mason Pfizer monkey virus or Simian Retroviruses; or from HTLV, human T cell leukemia virus type 1 and 2; or from Spumaviruses like, Simiam foamy virus, Human foamy virus, or feline syncytium-forming virus; or from G-protein of vesicular stomatitis virus (VSV-G).

5. The retroviral lentivirus based vector system according to claim 3 or 4, wherein the vector is derived from SIV and the Env is derived from SIV or an amphotropic, polytropic or xenotropic murine leukemia virus or from vesicular stomatitis virus (VSV-G-protein).

6. A retroviral particle comprising a retroviral lentivirus based vector according to any of the preceding claims 1 to 5.

7. The retroviral particle according to claim 6 obtainable by transfecting a packaging cell of the lentivirus based vector system according to any of the preceding claims 3 to 5 with the lentivirus based vector according to any of the preceding claims 1 to 5.

8. A retroviral provirus produced by infection of target cells with the retroviral particle according to claim 6 or 7.

9. mRNA of a retroviral provirus according to claim 8.

10. RNA of the retroviral lentivirus based vector according to any of the preceding claims 1 to 5.

11. cDNA of the RNA according to claim 10.

12. A host cell infected with the retroviral particle according to claim 6 or 7.

13. The retroviral particle according to claim 6 or 7 and/or the lentivirus based vector system according to any of the preceding claims 3 to 5 and/or the lentivirus based vector according to any of the preceding claims 1 to 5 for use in the treatment of a central nervous system disease or disorder or a metabolic liver disease or any other relevant disease or disorder.

14. A pharmaceutical composition containing a therapeutically effective amount of the retroviral particle according to claim 6 or 7 and/or the retroviral lentivirus based vector system according to any of the preceding claims 3 to 5.

15. Use of the lentivirus vector according to any of the preceding claims 1 to 5 and/or of the retroviral lentivirus based vector system according to any of the preceding claims 3 to 5 and/or of the retroviral particle according to claim 6 or 7 for producing a pharmaceutical composition for gene therapy.

16. The use according to claim 15 for the treatment of a central nervous system disease or disorder or a metabolic liver disease or any other relevant disease or disorder.

17. A method for introducing homologous and/or heterologous nucleotide sequences into target cells comprising infecting the target cells with retroviral particles according to claim 6 or 7.

18. A method of treating a central nervous system disorder or disease or metabolic liver disease or any other relevant disease or disorder of an animal including a human, which method comprises administering to a person in need thereof a therapeutically effective amount of the retroviral vector system according to any of the preceding claims 3 to 5 and/or of the retroviral particle according to claim 6 or 7.

19. A method of immunising, by vaccination or therapeutic vaccination, an animal including a human, against lentivirus infection, which method comprises administering to a person in need thereof a therapeutically effective amount of the retroviral vector system according to any of the preceding claims 3 to 5 and/or of the retroviral particle according to claim 6 or 7.

20. The method of claim 19 wherein the lentivirus infection is HIV or SIV or HTLV.
